(19) United States
(12) Patent Application Publication
(10) Pub. No.: US 2010/0297218 A1
(43) Pub. Date: Nov. 25, 2010

(54) TISSUE ADHESIVE COMPOSITIONS AND METHODS THEREOF

(75) Inventors: Glen Gong, San Francisco, CA (US); Charles Wartchow, Palo Alto, CA (US); Alison Schlosser, Los Altos, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: PneumRx, Inc., Mountain View, CA (US)

(21) Appl. No.: 12/602,468

(22) PCT Filed: Sep. 19, 2007
(86) PCT No.: PCT/US07/78932

§ 371 Date: Aug. 3, 2010

Related U.S. Application Data
(60) Provisional application No. 60/826,242, filed on Sep. 20, 2006.

(51) Int. Cl. A61K 38/38; A61K 38/00; A61K 9/127; A61P 43/00

(52) U.S. Cl. 424/450; 514/1.1; 514/15.2

(57) ABSTRACT

The present invention relates to composition and method for an adhesive composition. In particular, the present invention relates to a composition of an adhesive composition containing a protein component and an aldehyde component where the aldehyde component further comprises of a thickening agent. The adhesive composition is used for adhering to a tissue in various applications. The adhesive composition can be used for marking a location within the human body, such as a tumor.

### CROSS-REFERENCE

This application claims the benefit of U.S. Provisional Application No. 60/826,242, filed Sep. 20, 2006, which is incorporated herein by reference in its entirety.

### BACKGROUND OF THE INVENTION

The present invention relates to compositions and methods for an adhesive composition. In particular, the present invention relates to a composition of an adhesive composition containing a protein component and an aldehyde component where the aldehyde component further comprises a thickening agent. The adhesive composition is used for adhering to a tissue in various applications.

A variety of techniques have been used to bond or seal the tissue. Such techniques include sutures, staples, tapes and bandages. Sutures have customarily been used to repair tissue breaks. However, suturing can entail prolonged surgical time and surgical skill, especially in the presence of extensive injuries. The suture can produce different levels of tension in the tissue. This can lead to topographic distortions when stress is placed on certain areas of the tissue. Loose sutures can harbor bacteria and cause local inflammation and tissue necrosis as a prelude to infection and endophthalmitis. Suture removal can further add additional stress on the patient.

Tissue adhesive is another technique used to bond or seal the tissue. Some of the examples of the tissue adhesives include cyanoacrylates, gelatin-formaldehyde compositions, and fibrin based glues. Cyanoacrylate adhesive has limited use in the internal applications since the adhesive requires a dry field and is non-absorbable by the tissue. The polymerization of the cynoacrylate adhesive on the surface of the tissue may tend to be exothermic and can lead to adverse tissue response. Gelatin-formaldehyde compositions can be toxic since formaldehyde is a hazardous material. See, U.S. Pat. No. 5,385,606 to Kowanko for Adhesive Composition and Method; U.S. Pat. No. 6,849,262 to Ollerenshaw et al. for Vascular Coating Composition; and U.S. Pat. No. 6,372,229 to Ollerenshaw et al. for Vascular Coating Composition.

Fibrin glues use blood products (fibrinogen and co-factors) which can be obtained from multiple human donors. Fibrin glues utilize a natural process of blood clot formation to generate an adhesive or sealant composition. Fibrin glue is comprised of two components. One of the components is a solution of fibrinogen and blood clotting factors such as factor XIII, and the other component is primarily a solution of thrombin and calcium ion. Two components are combined to form an artificial blood clot. It can therefore present an inherent risk of transmitting diseases to the patient. Further, fibrin glues have low strength (generally less than 50 g/cm²) and relatively slow set up time.

In view of the foregoing, it would be desirable to have a composition and method for providing an effective and less toxic adhesive composition.

### SUMMARY OF THE INVENTION

One aspect of the present invention relates to an adhesive composition, comprising a protein component having a first viscosity, and an aldehyde component having a second viscosity, where the aldehyde component further comprises a thickening agent. In some embodiments, the protein component can be derived from a mammalian source. The mammalian sources include, for example, human, bovine, bison, ovine, and porcine. In other embodiments, the protein component can be derived from a recombinant source. In some embodiments, the protein component is a serum albumin. In some embodiments, the aldehyde component is, for example, a dialdehyde or a polyaldehyde. In some embodiments, the dialdehyde is a glutaraldehyde. In still other embodiments, the protein component is a serum albumin and the aldehyde component is a glutaraldehyde. The protein component can comprise more than 15% protein monomer. In some embodiments, the thickening agent increases the second viscosity of the aldehyde component by at least about 5%. The thickening agents include, for example, dextran, carboxymethyl cellulose, polyethylene glycol, liposomes, proliposomes, glycerol, starch, carbohydrates, povidone, polyethylene oxide, and polyvinyl alcohol. In some embodiments, the thickening agent is dextran, polyethylene glycol or carboxymethyl cellulose. In still other embodiments, the thickening agent is dextran. In some embodiments, the thickening agent can comprise at least about 0.5% of the composition. The thickening agent can alter a gel time of the composition.

Some embodiments of the aforementioned aspect of the present invention further comprise a radiopaque material. The radiopaque material includes, for example, bismuth oxide (Bi₂O₃), zinc oxide (ZnO), barium sulfate (BaSO₄) lanthanum oxide (La₂O₃), cerium oxide (CeO₂), terbium oxide, ytterbium oxide, neodymium oxide, zirconia (ZrO₂), strontia (SrO), tin oxide (SnO₂), radiopaque glass and silicate glass. The radioopaque glass includes, for example, barium silicate, silico-alumino barium or strontium containing glass. The silicate glass includes, for example, barium or strontium containing glass.

Another aspect of the present invention relates to an adhesive composition comprising a protein component having a first viscosity, and an aldehyde component having a second viscosity, where the composition comprises between about 1-26% protein concentration. In some embodiments, the protein component can comprise between about 1-75% protein concentration. In some embodiments, the protein component is a serum albumin. In some embodiments, the protein component and the aldehyde component are present in a ratio of from 6:1 to 1:6. In still other embodiments, the protein component and the aldehyde component are present in the ratio of 1:1. The protein component can also comprise more than 15% protein monomer.

Another aspect of the present invention relates to an adhesive composition comprising a mixture of a serum albumin component having a first viscosity, and an aldehyde component having a second viscosity, wherein the composition comprises between about 1-26% of the serum albumin concentration. In some embodiments, the serum albumin component is derived from porcine. In some embodiments, the aldehyde component is a glutaraldehyde. In some embodiments, the aldehyde composition further comprises a thickening agent. The thickening agent includes, for example, dextran, polyethylene glycol or carboxymethyl cellulose. In some embodiments, the second viscosity of the aldehyde component approximates the first viscosity of the serum albumin component. In some embodiments, the serum albumin component comprises between about 1-75% serum albumin concentration. In still other embodiments, the serum albumin component and the aldehyde component are present in a ratio of from 6:1 to 1:6. The protein component can also comprise more than 15% protein monomer.

Yet another aspect of the present invention relates to a method for causing adhesion of tissue, comprising mixing a first composition comprising a protein component having a first viscosity with a second composition comprising an aldehyde component having a second viscosity to obtain an adhesive composition and adjusting the second viscosity of the second composition with a thickening agent; introducing the adhesive composition to a tissue; and allowing the adhesive composition to adhere to the tissue. In some embodiments, the protein component is derived from porcine. In some embodiments, the protein component is a serum albumin. In some embodiments, the aldehyde component is a dialdehyde or a polyaldehyde. In still other embodiments, the aldehyde component is a glutaraldehyde. In some embodiments, the protein component is a serum albumin and the aldehyde component is a glutaraldehyde. In some embodiments, the first viscosity approximates the second viscosity. In still other embodiments, the first viscosity matches the second viscosity. The thickening agent includes, for example, dextran, carboxymethyl cellulose, polyethylene glycol, liposomes, proliposomes, glycerol, starch, carbohydrates, povidone, polyethylene oxide, and polyvinyl alcohol. In some embodiments, the thickening agent is dextran. In some embodiments, the thickening agent comprises at least about 0.5% of the adhesive composition. The thickening agent can alter a gel time of the adhesive composition. In some embodiments, the adhesive composition comprises between about 1-26% protein concentration. In some embodiments, the protein component and the aldehyde component are present in a ratio of 6:1 to 1:6 in the final composition.

Some embodiments of the aforementioned aspect of the present invention further comprise a radiopaque material. The radiopaque material includes, for example, bismuth oxide (Bi₂O₃), zinc oxide (ZnO), barium sulfate (BaSO₄) lanthanum oxide (La₂O₃), cerium oxide (CeO₂), terbium oxide, ytterbium oxide, neodymium oxide, zirconia (ZrO₂), strontia (SrO), tin oxide (SnO₂), radiopaque glass and silicate glass. The radioopaque glass includes, for example, barium silicate, silico-alumino barium or strontium containing glass. The silicate glass includes, for example, barium or strontium containing glass.

Another aspect of the present invention relates to a method for marking a location within the human body, comprising providing a first component comprising a protein having a first viscosity and a second component comprising an aldehyde having a second viscosity; mixing the first component with the second component resulting in a target composition; introducing the target composition to a location; and marking a site of the location with the target composition. In some embodiments, the target composition is a palpable material. In some embodiments, the protein is a serum albumin derived from porcine. In some embodiments, the aldehyde is a glutaraldehyde. In still other embodiments, the first viscosity of the first component or the second viscosity of the second component is altered prior to the mixing step.

Yet another aspect of the present invention relates to a process of making an adhesive composition, comprising providing a first protein component with a first viscosity; providing a second aldehyde component with a second viscosity; adjusting the first viscosity, the second viscosity or both; and mixing the first component with the second component to obtain an adhesive composition. In some embodiments, the protein component is a serum albumin derived from porcine. In some embodiments, the aldehyde component is a glutaraldehyde. In some embodiments, the first viscosity of the first component approximates the second viscosity of the second component.

These and other features and advantages of the present invention will be understood upon consideration of the following detailed description of the invention.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a flow chart depicting some of the embodiments of the compositions and methods as provide herein; and

FIGS. 2A-B illustrate a delivery device with a dual chamber for holding the protein component and the aldehyde component prior to delivery, and a delivery cannula; FIG. 2B illustrates the mixing chamber and delivery trocar of the device.

### DETAILED DESCRIPTION OF THE INVENTION

The present invention provides compositions and methods for an adhesive composition. In particular, the adhesive composition comprises of a protein component and an aldehyde component wherein the aldehyde component further comprises of a thickening agent. Any component of the composition can be a percentage of the composition assessed by weight, weight-to-weight, weight-to-volume, or volume-to-volume. Additionally any component of the composition can further be comprised of components and/or sub-components having a percentage assessed by weight, weight-to-weight, weight-to-volume, or volume-to-volume. Percentages for a composition, component, or sub-component can be determined before or after a user mixes the components or sub-components.

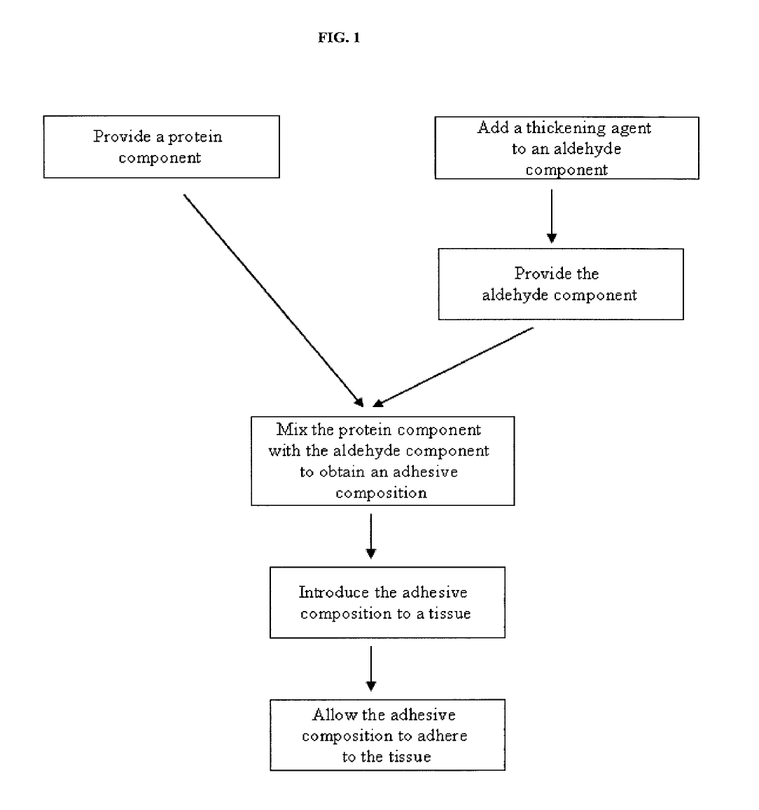

Some of the embodiments of the present invention are depicted in FIG. 1. It shall be understood that the invention includes other methods not explicitly set forth herein. A protein component may be provided. A thickening agent may be added to the aldehyde component and the aldehyde component may then be provided. The protein component and the aldehyde component may be mixed to obtain an adhesive composition. The adhesive composition may be then introduced to a tissue. The adhesive composition may be allowed to cure and adhere to the tissue.

Without limiting the scope of the invention, the steps can be performed in other alternate orders and/or one or more steps may be skipped. For example, the thickening agent may be added to the aldehyde component and the aldehyde component may be provided. A protein component may then be provided. The aldehyde component and the protein component are then mixed to obtain an adhesive composition. Alternatively, the protein component and the aldehyde component may be introduced directly to the tissue such that the adhesive composition is formed on the surface of the tissue.

I. Adhesive Composition

A. Protein Component in the Adhesive Composition

In some embodiments, the protein component in the adhesive composition, as provided herein, can be obtained from various mammalian sources, such as, for example, human, bovine (genus bos), bison (genus bison), ovine (genus ovis), porcine (genus sus), or other vertebrates. Alternatively, the protein component can be derived from a recombinant source such as, plant, non-animal source, bacteria (e.g., E. coli), yeast (e.g., Pichia pastoris, and Saccharomyces cerevisiae), mammalian cell, insect cell expression vector system, transgenic animals, etc. The protein component in the adhesive composition as provided herein is derived from a porcine source. The porcine source may not be infected with bacteria or viruses found in humans and other animals. For example, human blood can be infected with HIV and bovine blood can be infected with bacterial and viral impurities. Hence, the porcine source can provide a safe and non-toxic source of protein for the adhesive composition.

As will be appreciated by those skilled in the art, the genus mammalian source as provided above can include the entire sub genus and species that fall within the respective genus. For example, the genus sus (porcine) includes all the species that fall within the genus sus. Examples of the species of the genus sus include, but are not limited to, Sus barbatus; Sus bucculentus; Sus cebifrons; Sus celebensis; Sus daelius; Sus heureni; Sus philippensis; Sus salvanius; Sus scrofa; Sus timoriensis; Sus verrucosus; and Sus habeoncosus. The genus bos includes, but is not limited to, subgenus bos including bos taurus and bos aegyptiacus; subgenus bibos including bos frontalis and bos javanicus; subgenus novibos including bos sauveli, and; subgenus poephagus including bos grunniens. The species bos taurus, includes, but is not limited to, bos indicus; and the bos primigenius. Other species of the genus bos include bos gaurus laosiensis, bos gaurus gaurus, bos gaurus readei, bos gaurus hubbacki, and bos gaurus frontalis. The genus bison includes species such as, bison latifrons, bison antiquus, bison occidentalis, bison priscus, bison bison, bison bison bison, bison bison athabascae, bison bonasus, bison bonasus bonasus, bison bonasus caucasicus, and bison bonasus hungarorum.

The protein component in the adhesive composition, as provided herein, can be derived from blood plasma or serum of any of the mammalian sources. Examples of the techniques for deriving the protein component from blood plasma or serum include, but are not limited to, drying, evaporation, precipitation, amplification, fractionation, heat shock, reverse osmosis, nanofiltration, ultrafiltration, microfiltration, sedimentation, centrifugation, electrodialysis, dilution, adjustment of pH, addition of preservatives and calibrants, addition of denaturants, desalting of samples, concentration, extraction and purification. The protein component can be prepared and/or purified by techniques such as, ion exchange chromatography, high-performance liquid chromatography (HPLC), size exclusion chromatography (SEC), mass spectrometry (MS), metal ion affinity chromatography, gel filtration, hydrophobic chromatography, chromatofocusing, adsorption chromatography, isoelectric focusing and the like. After purification, the protein component can be further analyzed for properties such as, viscosity, and osmolality. It shall be understood that methods for deriving, purifying and/or analyzing the protein component are known in the art and are within the scope of the present invention.

The protein component as provided herein can comprise a purified protein or a mixture of proteins. The protein component may comprise a mixture of monomeric, dimeric, and/or polymeric protein contents. The protein component may further comprise other plasma proteins, endotoxins, metal ions, or albumin aggregates. In some embodiments, the protein component in the adhesive composition is substantially free of dimeric proteins, polymeric proteins, other plasma proteins, endotoxins, metal ions, albumin aggregates or any decomposed matters. In some embodiments, the protein component substantially consists of a protein with high monomer content.

The protein component of an adhesive composition can itself be further comprised of components and/or sub-components. Thus, the protein component can be described in terms of weight, weight-to-weight, weight-to-volume, or volume-to-volume, either before or after mixing.

In some embodiments, the protein component comprises at least about 50% monomeric protein; at least about 55% monomeric protein; at least about 60% monomeric protein; at least about 70% monomeric protein; at least about 80% monomeric protein; at least about 85% monomeric protein; at least about 90% monomeric protein; at least about 95% monomeric protein; at least about 99% monomeric protein; or at least about 99.99% monomeric protein. In some embodiments, the protein component comprises at least about 50% monomeric protein. In still embodiments, the protein component comprises at least about 90% monomeric protein.

The protein in the protein component can be a human or animal-derived serum albumin, or ovalalbumin. The protein can also be a commercial plasma extender such as Plasma-Plex or Plasmanate which may contain reconstituted solutions of about 5% plasma protein by weight or other appropriate solutions. In some embodiments, the protein in the protein component is serum albumin derived from a porcine source.

The protein component can comprise about 1-90% protein concentration. In some embodiments, the protein component comprises of about 1-75% protein concentration. In some embodiments, the protein component comprises of about 5-75% protein concentration; about 10-75% protein concentration; about 20-75% protein concentration; about 30-75% protein concentration; about 40-75% protein concentration; about 50-75% protein concentration; or about 60-75% protein concentration.

The adhesive composition as provided herein can comprise a low protein concentration. The low protein concentration can reduce the toxicity of the adhesive composition in the human or animal body. In some embodiments, the adhesive composition comprises at least about 1% protein concentration; at least about 5% protein concentration; at least about 8% protein concentration; at least about 10% protein concentration; at least about 12% protein concentration; at least about 15% protein concentration; at least about 18% protein concentration; at least about 20% protein concentration; at least about 22% protein concentration; at least about 24% protein concentration; at least about 26% protein concentration; or at least about 30% protein concentration. In some embodiments, the adhesive composition comprises about 1-26%, about 5-20%, or about 10-20% protein concentration.

In some embodiments, the adhesive composition comprises at least about 1% serum albumin concentration; at least about 5% serum albumin concentration; at least about 8% serum albumin concentration; at least about 10% serum albumin concentration; at least about 12% serum albumin concentration; at least about 15% serum albumin concentration; at least about 18% serum albumin concentration; at least about 20% serum albumin concentration; at least about 22% serum albumin concentration; at least about 24% serum albumin concentration; at least about 26% serum albumin concentration; or at least about 30% serum albumin concentration. In some embodiments, the adhesive composition comprises about 1-26%, about 5-20%, or about 10-20% of the serum albumin concentration.

B. Aldehyde Component in the Adhesive Composition

The aldehyde component in the adhesive composition as provided herein can be any biocompatible aldehyde with low toxicity. In particular, the aldehyde component includes a di-aldehyde, a polyaldehyde or a mixture thereof. The examples of the aldehyde include, but are not limited to, glyoxal, chondroitin sulfate aldehyde, succinaldehyde, glutaraldehyde, and malealdehyde. In some embodiments, the aldehyde component is glutaraldehyde. Other suitable aldehydes which have low toxicity include multifunctional aldehydes derived from naturally-occurring substances, e.g., dextrandialdehyde, or saccharides. The aldehyde component can be an aldehyde product obtained by an oxidative cleavage of carbohydrates and their derivatives with periodate, ozone or the like. The aldehyde may optionally be pre-treated with heat. See US 2004/0081676 by Schankereli for Biocompatible phase invertable proteinaceous compositions and methods for making and using the same. The aldehyde component can be analyzed for properties such as, viscosity, and osmolality.

The aldehyde component of an adhesive composition can itself be further comprised of components and/or sub-components. Thus, the aldehyde component can be described in terms of weight, weight-to-weight, weight-to-volume, or volume-to-volume, either before or after mixing.

In some embodiments, the aldehyde component comprises of about 1-90% aldehyde concentration. In some embodiments, the aldehyde component comprises of about 1-75% aldehyde concentration. In some embodiments, the aldehyde component comprises of about 5-75% aldehyde concentration; about 10-75% aldehyde concentration; about 20-75% aldehyde concentration; about 30-75% aldehyde concentration; about 40-75% aldehyde concentration; about 50-75% aldehyde concentration; or about 60-75% aldehyde concentration.

The adhesive composition can comprise at least about 1% aldehyde concentration; at least about 5% aldehyde concentration; at least about 10% aldehyde concentration; at least about 20% aldehyde concentration; at least about 30% aldehyde concentration; at least about 40% aldehyde concentration; at least about 50% aldehyde concentration; at least about 60% aldehyde concentration; at least about 70% aldehyde concentration; at least about 80% aldehyde concentration; at least about 90% aldehyde concentration; or at least about 99% aldehyde concentration. In some embodiments, the adhesive composition comprises of about 1-30%, about 25-75%, about 50-75% or about 75-99% aldehyde concentration.

In some embodiments, the adhesive composition comprises of at least about 1% glutaraldehyde concentration; at least about 5% glutaraldehyde concentration; at least about 8% glutaraldehyde concentration; at least about 10% glutaraldehyde concentration; at least about 20% glutaraldehyde concentration; at least about 30% glutaraldehyde concentration; at least about 40% glutaraldehyde concentration; at least about 50% glutaraldehyde concentration; at least about 60% glutaraldehyde concentration; at least about 70% glutaraldehyde concentration; at least about 80% glutaraldehyde concentration; at least about 90% glutaraldehyde concentration; or at least about 99% glutaraldehyde concentration. In some embodiments, the adhesive composition comprises about 1-30%, about 25-75%, about 50-75% or about 75-99% glutaraldehyde concentration.

C. Thickening Agent or Thinning Agent in the Adhesive Composition

In some embodiments, the adhesive composition of the present invention can further comprise a thickening agent. The examples of the thickening agent include, but are not limited to, dextran, carboxymethyl cellulose, polyethylene glycol, liposomes, proliposomes, glycerol, starch, carbohydrates, povidone, polyethylene oxide, and polyvinyl alcohol. In some embodiments, the thickening agent is dextran.

The thickening agent can be added to the aldehyde component before the aldehyde component is mixed with the protein component to make the adhesive composition. The thickening agent can alter the viscosity of the aldehyde component and thereby alter the gel time of the adhesive composition. Alteration of the viscosity of the aldehyde component includes any modification of the viscosity of the composition. Alteration of the gel time of the aldehyde component includes any modification of the gel time of the composition. For example, alteration includes increase or decrease in the gel time of the adhesive composition. The gelling of the adhesive composition includes a substantial increase in a viscosity that can be measured using a rheometer or viscometer instrument. The increase in the viscosity includes transition from a fluid state to a solid or thicksotropic state. For example, the thickening agent can increase the viscosity of the aldehyde component and hence, decrease the gel time of the adhesive composition.

The amount of the thickening agent added to the aldehyde component can be varied depending on the gel time needed for the adhesive composition. A quick-setting adhesive with a short gel time can be used to stop tissues from bleeding or to seal off tissues from their surroundings. A slower set adhesive with a long gel time allows the user to apply the adhesive to two surfaces and then approximate the tissues to effectively glue the surfaces together. Hence, if a shorter gel time (such as, less than a minute) is needed for the adhesive composition then a higher amount of thickening agent may be added to the aldehyde component. Alternatively, if a longer gel time (such as, few minutes to hours) is desired for the adhesive composition then a smaller amount of thickening agent may be added to the aldehyde component. Such optimization of the concentration of the thickening agent is within the skill of those skilled in the art.

In some embodiments, the aldehyde component comprises at least about 0.5% thickening agent; at least about 2% thickening agent; at least about 5% thickening agent; at least about 10% thickening agent; at least about 20% thickening agent; at least about 30% thickening agent; at least about 40% thickening agent; at least about 50% thickening agent; at least about 60% thickening agent; at least about 70% thickening agent; at least about 80% thickening agent; or at least about 90% thickening agent.

The adhesive composition can comprise at least about 0.5% of the thickening agent. In some embodiments, the adhesive composition comprises at least about 1% thickening agent concentration; at least about 5% thickening agent concentration; at least about 10% thickening agent concentration; at least about 20% thickening agent concentration; at least about 30% thickening agent concentration; at least about 40% thickening agent concentration; at least about 50% thickening agent concentration; at least about 60% thickening agent concentration; at least about 70% thickening agent concentration; at least about 80% thickening agent concentration; or at least about 90% thickening agent concentration. In some embodiments, the adhesive composition comprises at least about 0.5%-10%, at least about 0.5%-25%, or at least about 0.5%-50% thickening agent concentration.

The amount of thickening agent added to the aldehyde component can be varied depending on the desired increase in the viscosity of the aldehyde component. In some embodiments, the thickening agent increases the viscosity of the aldehyde component such that the viscosity of the aldehyde component matches or approximates the viscosity of the protein component. Such matching of the aldehyde component viscosity with the protein component viscosity can enhance the mixing efficiency of the two components in the process of making the adhesive composition.

In some embodiments, the thickening agent increases the viscosity of the aldehyde component by at least about 5%. In some embodiments, the thickening agent increases the viscosity of the aldehyde component by at least about 8%; at least about 10%; at least about 15%; at least about 20%; at least about 25%; at least about 30%; at least about 40%; at least about 50%; at least about 60%; at least about 70%; at least about 80%; or at least about 90%.

The thinning agent can be added to the protein component before the protein component is mixed with the aldehyde component to make the adhesive composition. The thinning agent can alter the viscosity of the protein component and thereby alter the gel time of the adhesive composition. For example, the thinning agent can decrease the viscosity of the protein component and hence, increase the gel time of the adhesive composition. The amount of thinning agent added to the protein component can be varied depending on the gel time needed for the adhesive composition. For example, if a longer gel time (such as, few minutes to hours) is desired for the adhesive composition then a higher amount of thinning agent may be added to the protein component. Alternatively, if a shorter gel time (such as, less than a minute) is needed for the adhesive composition then a lower amount of thinning agent may be added to the protein component. Such optimization of the concentration of the thinning agent is within the skill of those skilled in the art.

In some embodiments, the protein component comprises at least about 0.5% thinning agent; at least about 2% thinning agent; at least about 5% thinning agent; at least about 10% thinning agent; at least about 20% thinning agent; at least about 30% thinning agent; at least about 40% thinning agent; at least about 50% thinning agent; at least about 60% thinning agent; at least about 70% thinning agent; at least about 80% thinning agent; or at least about 90% thinning agent.

The adhesive composition can comprise at least about 0.5% of the thinning agent. In some embodiments, the adhesive composition comprises at least about 1% thinning agent; at least about 5% thinning agent; at least about 10% thinning agent; at least about 20% thinning agent; at least about 30% thinning agent; at least about 40% thinning agent; at least about 50% thinning agent; at least about 60% thinning agent; at least about 70% thinning agent; at least about 80% thinning agent; or at least about 90% thinning agent. In some embodiments, the adhesive composition comprises at least about 0.5%-10%, at least about 0.5%-25%, or at least about 0.5%-50% thinning agent.

The amount of thinning agent added to the protein component can be varied depending on the desired decrease in the viscosity of the protein component. In some embodiments, the thinning agent decreases the viscosity of the protein component such that the viscosity of the protein component matches or approximates the viscosity of the aldehyde component. Such matching of the aldehyde component viscosity with the protein component viscosity can enhance the mixing efficiency of the two components in the process of making the adhesive composition.

In some embodiments, the thinning agent decreases the viscosity of the protein component by at least about 5%. In some embodiments, the thinning agent decreases the viscosity of the protein component by at least about 8%; at least about 10%; at least about 15%; at least about 20%; at least about 25%; at least about 30%; at least about 40%; at least about 50%; at least about 60%; at least about 70%; at least about 80%; or at least about 90%.

D. Other Additives in the Adhesive Composition

The adhesive composition as provided herein can optionally contain other additives. These additives may be added to the protein component or the aldehyde component prior to their mixing. Alternatively, these materials may be added to the adhesive composition after the mixing of the protein component and the aldehyde component.

Without limiting the scope of the present invention, some of the examples of the additives are as below.

In some embodiments, the adhesive composition optionally comprises a radiopaque material. The examples of the radiopaque material include, but are not limited to, heavy metals, oxides, sulfates, ceramics and fluorides that are not hazardous such as bismuth oxide (Bi₂O₃), zinc oxide (ZnO), barium sulfate (BaSO₄) lanthanum oxide (La₂O₃), cerium oxide (CeO₂), terbium oxide, ytterbium oxide, neodymium oxide, zirconia (ZrO₂), strontia (SrO), tin oxide (SnO₂), and radiopaque glasses such as barium silicate, silico-alumino barium or strontium containing glasses and silicate glasses containing barium or strontium. In some embodiments, the radioopaque material comprises at least about 0.001%; at least about 0.05%; at least about 0.1%; at least about 0.2%; at least about 0.5%; at least about 1%; at least about 2%; at least about 5%; at least about 8%; or at least about 10% of the adhesive composition.

The adhesive composition as provided herein can optionally contain additives such as, but not limited to, water, buffer, saline solution, neutral salt, carbohydrate, fiber, miscellaneous biological material, wetting agent, antibiotics, preservative, dye, chitosans, thickening agent, thinning agent, fibrinogen, polymer such as polyethylene glycol or combination thereof. Polymers include synthetic polymers such as, polyamides, polyesters, polystyrenes, polyacrylates, vinyl polymers (e.g., polyethylene, polytetrafluoro-ethylene, polypropylene and polyvinyl chloride), polycarbonates, polyurethanes, poly dimethyl siloxanes, cellulose acetates, polymethyl methacrylates, ethylene vinyl acetates, polysulfones, nitrocelluloses and similar copolymers. Polymers further include biological polymers which can be naturally occurring or produced in vitro by fermentation and the like. Biological polymers include, without limitation, collagen, elastin, silk, keratin, gelatin, polyamino acids, polysaccharides (e.g., cellulose and starch) and copolymers thereof.

Flexibilizers can be included in the adhesive composition to provide flexibility to the adhesive bond upon curing. Flexibilizers may be naturally occurring compositions. Suitable flexiblizers include synthetic and natural rubbers, synthetic polymers, natural non-native biocompatible proteins (such as exogenous (i.e., non-native) collagen and the like), glycosaminoglycans (GAGs) (such as haluronin and chondroitin sulfate), and blood components (such as fibrin, fibrinogen, albumin and other blood factors).

The adhesive composition as provided herein can optionally include salts and/or buffers. Examples of the salt include, but are not limited to, sodium chloride, potassium chloride and the like. Suitable buffers can include, for example, ammonium, phosphate, borate, bicarbonate, carbonate, cacodylate, citrate, and other organic buffers such as tris(hydroxymethyl) aminomethane (TRIS), morpholine propanesulphonic acid (MOPS), and N-(2-hydroxyethyl) piperazine-N′(2-ethanesulfonic acid) (HEPES). Suitable buffers can be chosen based on the desired pH range for the adhesive composition.

Additional additives may be present in the adhesive composition to modify the mechanical properties of the composition. Some additives include, for example, fillers, softening agents and stabilizers. Examples of fillers include, but are not limited to, carbon black, metal oxides, silicates, acrylic resin powder, and various ceramic powders. Examples of softening agents include, but are not limited to, dibutyl phosphate, dioctylphosphate, tricresylphosphate, tributoxyethyl phosphates and other esters. Examples of stabilizers include, but are not limited to, trimethyldihydroquinone, phenyl-β-naphthyl amine, p-isopropoxydiphenylamine, diphenyl-p-phenylene diamine and the like.

II. Process of Making Adhesive Composition

Another aspect of the present invention relates to a process of making the adhesive composition by mixing the protein component and the aldehyde component to obtain an adhesive composition. In some embodiments, a thickening agent is added to the aldehyde component prior to mixing of the aldehyde component with the protein component. Alternatively, a thinning agent is added to the protein component prior to mixing of the protein component with the aldehyde component.

In some embodiments, the protein component and the aldehyde component are present in the adhesive composition in a ratio of from 6:1 to 1:6. In some embodiments, the protein component and the aldehyde component are present in a ratio of 6:1, 5:1, 4:1, 3:1, 2:1, 1:1, 1:2, 1:3, 1:4, 1:5, or 1:6. In some embodiments, the protein component and the aldehyde component are present in a ratio of 1:1.

The serum albumin component and the aldehyde component can be present in the adhesive composition in a ratio of from 6:1 to 1:6. In some embodiments, the serum albumin component and the aldehyde component are present in a ratio of 6:1, 5:1, 4:1, 3:1, 2:1, 1:1, 1:2, 1:3, 1:4, 1:5, or 1:6. In some embodiments, the serum albumin component and the aldehyde component are present in a ratio of 1:1.

The protein component, aldehyde component, and thickening agent/thinning agent can be filled in a separate container such as vial or syringe etc. Thus, the protein component, aldehyde component, and thickening agent/thinning agent can then be mixed according to the invention, just prior to the use. In some embodiments, the thickening agent can be mixed with the aldehyde component before filling the aldehyde component in the container. Alternatively, the protein component, aldehyde component, and thickening agent/thinning agent can be mixed to make the adhesive composition and the adhesive composition can be filled into the container.

Alternatively, each of the protein component, aldehyde component and thickening agent/thinning agent can be mixed separately with suitable additives and then separately filled in the container. Suitable additives can also be added to the adhesive composition before filling the adhesive composition into the container. The container can be filled in the aseptic conditions. The solutions can also be sterilized after filling.

The solution of the protein component, aldehyde component, thickening agent, or thinning agent can be lyophilized. The lyophilized protein component, aldehyde component, thickening agent, or thinning agent can be reconstituted with water or saline just prior to use. Alternatively, the solution of the adhesive composition can be lyophilized. The lyophilized adhesive composition can be reconstituted with water or saline just prior to use.

The protein component, aldehyde component, thickening agent, thinning agent or the adhesive composition may be filled in the container and sealed with packaging materials which may not allow transmission of oxygen.

Once the desired components are selected, the user mixes the components to form the desired composition.

III. Method of Use

Another aspect of the invention relates to introducing an adhesive composition at a target, or selected, location within the body, such as on tissue, organ, organ component, or cavernous component of an organism such that the adhesive composition adheres to the tissue, organ, organ component, or cavernous component. In particular, the present invention relates to a method for causing adhesion of tissue by mixing the protein component with the aldehyde component; adjusting the viscosity of the aldehyde component with a thickening agent; introducing the adhesive composition to a tissue; and allowing the adhesive composition to adhere to the tissue. Alternatively, the protein component and the aldehyde component are allowed to react on one or more surfaces of the tissue to be bonded.

In some embodiments of the present invention, the adhesion can be rapid and can take place in less than a minute, such as 10-20 seconds. In some embodiments, the adhesion of the adhesive composition may take place from 1-10 minutes, 10 minutes-1 hr, or 1-5 hr. The gel time of the adhesive composition can be optimized by altering the viscosity of the aldehyde component using the thickening agent. Alternatively, the gel time can be optimized by altering the viscosity of the protein component by using the thinning agent. The gel time of the adhesive compositions as provided herein can also be optimized by use of buffers having different pH values or buffers having different ionic strengths. The adhesion composition of the invention can provide bond/tear strength of about 300-1500 g/cm². The bond/tear strength and the gel time can be varied depending on the variables such as temperature, pH, viscosity of the protein component, viscosity of the aldehyde component or the like.

The adhesive composition of the present invention can provide strong and rapid bonding to a wide range of substrates of natural or synthetic origin, providing a broad range of possible applications. Thus, the adhesive composition of present invention can bond to living tissues, including muscle, skin, connective tissue, nerve tissue, vascular and cardiac tissues, adipose tissue, cartilage, bone, and the like. The adhesive composition can bond to corresponding cadaver tissues, which may be preserved or otherwise chemically treated. The adhesive composition can also bond to natural or synthetic materials such as, leather, rubber, Dacron®, or Teflon®, as well as to metals.

The adhesive composition as provided herein can be used for the attachment of surgical grafts and devices, wound closure, trauma repair, hemostasis, and the like in the practice of human or veterinary medicine. Non medical applications of the adhesive are also anticipated. The adhesive composition may be used to bind tissue together either as an adjunct to or as a replacement of sutures, staples, tapes and/or bandages. In some embodiments, the adhesive composition as provided herein may be used to prevent post-surgical adhesions. In this application, the adhesive composition can be applied and cured as a layer on surfaces of internal organs or tissues in order to prevent the formation of adhesions at a surgical site as the site heals. Additional applications of the subject adhesive composition include sealing tissues to prevent or control blood or other fluid leaks at suture or staple lines as well as to prevent or control air leaks in the pulmonary system. In another application, the subject adhesive composition may be used to attach skin grafts and to position tissue flaps or free flaps during reconstructive surgery. In still another application, this adhesive composition may be used to close gingival flaps in periodontal surgery.

In some embodiments, the adhesive composition can be used to attach prosthesis to a native support tissue within a patient. For example, a cardiac prosthesis, such as heart valve prosthesis or an annuloplasty ring, can be secured to the corresponding native support tissue (i.e., annulus) within the patient using the subject adhesive composition. Examples of prostheses include, but are not limited to, prosthetic hearts, prosthetic heart valves, annuloplasty rings, vascular and coronary and structural stents, vascular grafts or conduits, implantable vascular devices, anastomotic connectors, leads, pacing leads, guidewires, permanently in-dwelling percutaneous devices, vascular or cardiovascular shunts, dermal grafts for wound healing, surgical patches, neurological growth supports, Hickman catheters, and bone replacement grafts, such as joint replacement prostheses. Implantable vascular devices include, for example, vascular grafts and conduits, pacemakers, valved grafts, stents, heart valves, patches, and anastomotic connectors.

The adhesive composition can be used to stop a hemorrhage in general surgery, reconstruct nerve ruptures in neurosurgery, adhere skin and cartilage transplants and defects in plastic surgery, treat pneumothorax and/or fistulas in general or thoracic surgery, and support vascular and intestinal anastomoses in vascular and general surgery. The subject adhesive composition can be used in the treatment of chondral and osteochondral fractures, transplantation of chondral or osteochondral materials, treatment of osteochondritis dissecans, joint fractures, meniscal tears as well as ruptured ligaments, tendons, myotendineous junctions or muscles, cartilage transplantation, bone transplantation, ligament transplantation, tendon transplantation, chondral transplantation, chondro-osseous transplantation, osseous transplantation, skin graft fixation, grafting (repairing) nerves and blood vessels, patching vascular grafts, microvascular blood vessel anastomosis, and treatment of combinations of the tissue surfaces. Furthermore, the subject adhesive composition may also comprise materials such as, collagen, gelatin, fibrinogen, fibrin, macromolecules, cell-attachment proteins, growth factors, cells etc., in order to enhance and stimulate the healing processes.

The adhesive composition can be used in surgical settings such as, operating rooms, emergency rooms and the like. The physician, for example, can use the adhesive composition for implanting prosthesis or for adhering a patients' native tissue to itself. The adhesive composition can also be used in manufacturing settings. Manufacturing settings can include settings where medical devices are assembled. For example, in a heart valve prosthesis, one or more of the heart valve leaflets can be associated with the support structure by the subject adhesive composition. The adhesive composition can be applied to a supporting structure such as a stent or a conduit that supports prosthesis.

In yet another aspect, the invention provides a method of marking a location within the human or animal body by providing the subject adhesive composition to a site of the location within the human body and thereby marking the location. For example, the subject adhesive composition can be used to mark a tissue/biopsy site for malignant, benign, or other lesions. Typically, physicians obtain a tissue specimen or sample from a lesion, suspicious site, organ, etc. for pathological examination and analysis, in order to diagnose and treat medical conditions. Tissue samples may be taken during exploratory or typical open surgery procedures. The tissue samples can be taken percutaneously or endoscopically during biopsy or surgical procedures. In the case of malignant, benign, or other lesions, it can take several days to weeks, following tissue sampling, to compile the results of pathologic examination and testing and to determine an appropriate medical treatment before the surgery or biopsy. It can be difficult for the physician, surgeon, or clinician to locate the site of the lesion again, in order to perform subsequent therapy. To execute the required treatment the site where the sample(s) is taken needs to be accurately located.

The subject adhesive composition marks a site of the lesion in the human or animal body by adhering to the tissue at the site of the lesion. The adhesive composition polymerizes into a palpable material. Since the subject adhesive composition adheres to the tissue and then polymerizes in situ, it does not move away from the lesion site. The physician, surgeon, or clinician can then locate the site of the lesion by the touching, or palpating, the region. The polymerized material, unlike the lesion itself, is perceivable by touch and therefore locatable by the physician, surgeon or clinician. Once the physician locates the lesion using palpation, surgical excision of the lesion can be performed with the least amount of collateral tissue damage. The polymerized adhesive composition can be adsorbed slowly by the tissue by hydrolysis.

The adhesive composition of the present invention may be applied to tissue in a number of different ways. For example, the protein component and the aldehyde component may be mixed together and then applied using common applicators. Alternatively, the two components may be mixed together and then applied as spray.

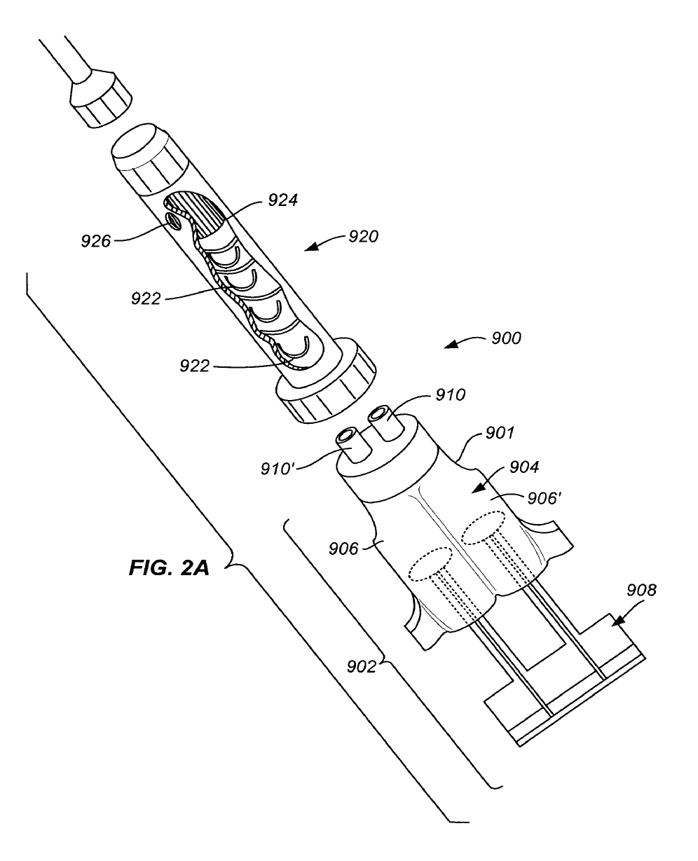

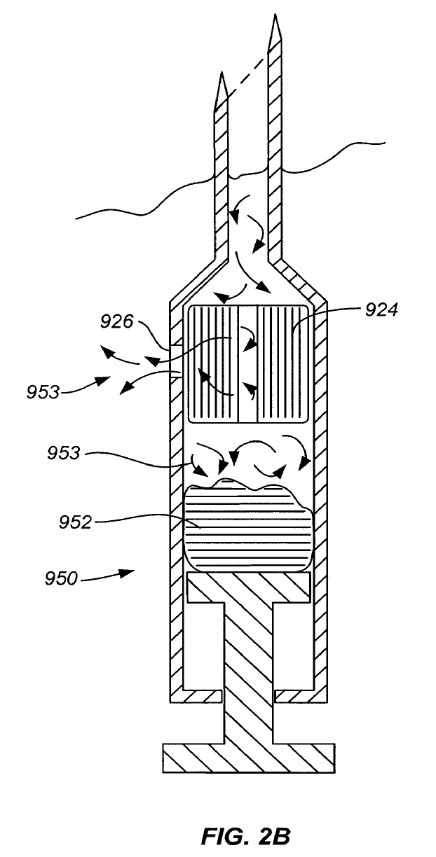

In some embodiments, the adhesive may be applied using a delivery device as shown in FIG. 2A-B (US Application No. 2006/0025815 to McGurk et al. for Lung Device With Sealing Features). FIG. 2A illustrates the adhesive delivery portion of the device 900. The proximal end 902 of the device 900 features the dual chamber 904 delivery housing 901. The adhesive delivery housing is separated into at least two chambers 906, 906′ in order to separate the protein component and the aldehyde component of the adhesive composition to be delivered. Thus, each chamber can comprise a component of the adhesive composition. The components of the adhesive composition can be delivered down the separate channels of the device 900. A plunger 908 is provided to advance the components down each chamber of the delivery housing. The protein component and the aldehyde component are advanced through separate sealed tips 910, 910′ in order to facilitate easy replacing of the stir chamber 920 in the event of a clog.

The adhesive delivery housing 901 is easily separable from the stir chamber 920 to facilitate replacement during a procedure. The stir chamber 920 receives the protein component and the aldehyde component from at least two ports of the adhesive delivery housing. Mixing elements or baffles 922 are provided to mix the components together as the components advance down the stir chamber 920. The mixing chamber can have prongs that interact with tips to break its seals when the mixing chamber is connected to the device.

The distal end of the stir chamber 920 features a porous plug filter 924 that enables air to escape the stir chamber 920 through an air bleed hole 926 located on the side of the stir chamber 920 at its distal end. Suitable filters include microfilters available from GenProbe. The filter properties are such that air can be dispersed through the filter transverse to the axis of the adhesive while the adhesive will be forced axially through the filter. FIG. 2B illustrates another delivery device having a plunger 950 advancing adhesive 952 through a chamber while air 953 is advanced through a porous plug filter 924 where it can exit through the air bleed hole 926 before the adhesive is delivered through the cannula into the target tissue.

In operation, the user deliver the tip of the delivery device to a target location within a patient; the user activates the mixing feature (e.g., by pressing the plunger) to begin mixing the components; the composition (i.e., the mixed components) is then delivered to the target location.

### EXAMPLES

### A. Pulmonary Sealant

### Example 1

A sealant composition was prepared comprising 40% albumin and 0.5% chitosan and a crosslinker solution containing 7.5% heat-processed glutaraldehyde and 20% dextran. The process followed for making the sealant was as described above. The lungs of an anaesthetized pig were exposed and deflated. Following this step, a portion of the upper lobe of the lung was transected and the cut site of the deflated lung was sealed by applying sealant from a dual chamber syringe with mixing tip and then re-inflated. The lung was evaluated for leakage by submersion in water. Evaluation of the lung for air leakage did not indicate any to be present, indicating the efficacy of the sealant. The composition successfully sealed the lung indicating the efficacy of the sealant.

### Example 2

A sealant was prepared comprising 30% albumin and 0.2% chitosan and a crosslinker solution containing 7.5% heat-processed glutaraldehyde and 20% dextran. The process followed for making the sealant was as described above. The lungs of an anaesthetized pig were exposed and deflated. Following this step, a portion of the upper lobe of the lung was transected and the cut site of the deflated lung was sealed by applying sealant from a dual chamber syringe with mixing tip and then re-inflated. The lung was evaluated for leakage by submersion in water. Evaluation of the lung for air leakage did not indicate any to be present, indicating the efficacy of the sealant. The composition successfully sealed the lung indicating the efficacy of the sealant.

### Example 3

A sealant was prepared comprising 38% albumin and 0.2% chitosan and a crosslinker solution containing 4.4% heat-processed glutaraldehyde and 20% dextran. The process followed for making the sealant was as described above. The lungs of an anaesthetized pig were exposed and deflated. Following this step, a portion of the upper lobe of the lung was transected and the cut site of the deflated lung was sealed by applying sealant from a dual chamber syringe with mixing tip and then re-inflated. The lung was evaluated for leakage by submersion in water. Evaluation of the lung for air leakage did not indicate any to be present, indicating the efficacy of the sealant. The composition successfully sealed the lung indicating the efficacy of the sealant.

### B. Pleural Adhesive

### Example 1

A sealant composition comprising 45% albumin and a crosslinker solution containing 7.5% heat-processed glutaraldehyde and 3% carboxymethyl cellulose, was prepared. The process followed for making the sealant was as described above. The visceral pleura surface of an anesthetized pig lung was exposed. The lung was then inflated insuring positive air pressure to physiological conditions. Sealant then was applied to the anterior portion of the pleural surface using a delivery catheter. Following 20 seconds, a section of chest wall containing parietal pleura was placed directly on top of the anterior portion of the visceral pleura. The sealant was then allowed to set for one minute. The adhesion of the two pleural surfaces was then evaluated by lifting up the chest wall (parietal pleura) only. If the two pleura surfaces were still attached then the adhesive was considered effective. The two pleural surfaces adhered together with the use of this composition.

### Example 2

A sealant composition comprising 40% albumin and 0.2% chitosan and a crosslinker solution containing 4% heat-processed glutaraldehyde and 20% dextran, was prepared. The process followed for making the sealant was as described above. The visceral pleura surface of an anesthetized pig lung was exposed. The lung was then inflated insuring positive air pressure to physiological conditions. Sealant then was applied to the anterior portion of the pleural surface using a delivery catheter. Following 20 seconds, a section of chest wall containing parietal pleura was placed directly on top of the anterior portion of the visceral pleura. The sealant was then allowed to set for one minute. The adhesion of the two pleural surfaces was then evaluated by lifting up the chest wall (parietal pleura) only. If the two pleura surfaces were still attached then the adhesive was considered effective. The two pleural surfaces adhered together with the use of this composition.

### Example 3

A sealant composition comprising 38% albumin and 0.2% chitosan and a crosslinker solution containing 7.5% heat-processed glutaraldehyde and 20% dextran, was prepared. The process followed for making the sealant was as described above. The visceral pleura surface of an anesthetized pig lung was exposed. The lung was then inflated insuring positive air pressure to physiological conditions. Sealant then was applied to the anterior portion of the pleural surface using a delivery catheter. Following 20 seconds, a section of chest wall containing parietal pleura was placed directly on top of the anterior portion of the visceral pleura. The sealant was then allowed to set for one minute. The adhesion of the two pleural surfaces was then evaluated by lifting up the chest wall (parietal pleura) only. If the two pleura surfaces were still attached then the adhesive was considered effective. The two pleural surfaces adhered together with the use of this composition.

### C. Vascular Sealant

### Example 1

A sealant composition comprising 40% albumin and 1.0% chitosan and a crosslinker solution containing 3.5% heat-processed glutaraldehyde and 20% glycerol, was prepared. The arteries of an anesthetized, anticoagulated rabbit were exposed. The artery of the left side was punctured with a catheter. Following removal of the catheter, the hole was closed using the sealant. Alternately, the artery of the right side was transected, and an anastomosis was created using a suture. An umbilical tape was partially looped around the vessel proximal to the surgery site to momentarily reduce blood flow. Sealant was then applied to the puncture site using a dual chamber syringe with mixing tip. Following two minutes, the pressure was released to expose the repair to the full systolic/diastolic pressure of the carotid artery. No leakage was found to be present from the wound site. Sealant was also applied to the partially leaking anastomotic site of the right side of the experimental model. Following two minutes it was observed that the leakage stopped.

### Example 2

A sealant comprising 38% albumin and 0.2% chitosan and a crosslinker solution containing 7.0% heat-processed glutaraldehyde and 6% povidone, was prepared. The arteries of an anesthetized, anticoagulated rabbit were exposed. The artery of the left side was punctured with a catheter. Following removal of the catheter, the hole was closed using the sealant. Alternately, the artery of the right side was transected, and an anastomosis was created using a suture. An umbilical tape was partially looped around the vessel proximal to the surgery site to momentarily reduce blood flow. Sealant was then applied to the puncture site using a dual chamber syringe with mixing tip. Following two minutes, the pressure was released to expose the repair to the full systolic/diastolic pressure of the carotid artery. No leakage was found to be present from the wound site. Sealant was also applied to the partially leaking anastomotic site of the right side of the experimental model. Following two minutes it was observed that the leakage stopped.

### Example 3

A sealant comprising 35% albumin and 1.0% chitosan and a crosslinker solution containing 4.4% heat-processed glutaraldehyde and 20% dextran, was prepared. The arteries of an anesthetized, anticoagulated rabbit were exposed. The artery of the left side was punctured with a catheter. Following removal of the catheter, the hole was closed using the sealant. Alternately, the artery of the right side was transected, and an anastomosis was created using a suture. An umbilical tape was partially looped around the vessel proximal to the surgery site to momentarily reduce blood flow. Sealant was then applied to the puncture site using a dual chamber syringe with mixing tip. Following two minutes, the pressure was released to expose the repair to the full systolic/diastolic pressure of the carotid artery. No leakage was found to be present from the wound site. Sealant was also applied to the partially leaking anastomotic site of the right side of the experimental model. Following two minutes it was observed that the leakage stopped.

### D. Dural Sealant

### Example 1

A sealant composition comprising 40% albumin and 5% Polyethylene glycol and a crosslinker solution containing 7.5% heat-processed glutaraldehyde and, was prepared. The process followed for making the sealant was as described above. Following a craniotomy, the exposed dura of a bovine model was incised. Incision of the dura resulted in retraction of the tissue. The retracted tissue was drawn together, again using temporary sutures such that the incised edges were opposed to one another. Sealant was applied with dual chamber syringe with mixing tip over the incision wound and the suture stays were released. The opposing edges of the incision wound remained aligned with one another, all three sealant compositions demonstrated adequate tenacity to resist the refractive forces of the dura. The head was lowered placing additional stress on the suture and the site was observed for failure of the sealant to hold the edges together. No failures were observed.

### Example 2

A sealant composition comprising 38% albumin and 0.2% hyaluronic acid and a crosslinker solution containing 4.4% heat-processed glutaraldehyde and 20% dextran, was prepared. The process followed for making the sealant was as described above. Following a craniotomy, the exposed dura of a bovine model was incised. Incision of the dura resulted in refraction of the tissue. The refracted tissue was drawn together, again using temporary sutures such that the incised edges were opposed to one another. Sealant was applied with dual chamber syringe with mixing tip over the incision wound and the suture stays were released. The opposing edges of the incision wound remained aligned with one another, all three sealant compositions demonstrated adequate tenacity to resist the retractive forces of the dura. The head was lowered placing additional stress on the suture and the site was observed for failure of the sealant to hold the edges together. No failures were observed.

### Example 3

A sealant composition comprising 35% albumin and 1.0% hyaluronic acid and a crosslinker solution containing 4.4% heat-processed glutaraldehyde and 15% dextran, was prepared. The process followed for making the sealant was as described above. Following a craniotomy, the exposed dura of a bovine model was incised. Incision of the dura resulted in retraction of the tissue. The retracted tissue was drawn together, again using temporary sutures such that the incised edges were opposed to one another. Sealant was applied with dual chamber syringe with mixing tip over the incision wound and the suture stays were released. The opposing edges of the incision wound remained aligned with one another, all three sealant compositions demonstrated adequate tenacity to resist the retractive forces of the dura. The head was lowered placing additional stress on the suture and the site was observed for failure of the sealant to hold the edges together. No failures were observed.

### E. Tissue/Biopsy Site Marker

An adhesive composition is for use as a tissue/bioptsy site marker is prepared using two solutions; a protein solution of purified porcine serum albumin (PSA) with chitosan (Solution “A”), a hemostatic agent, and a solution of processed glutaraldehyde (PGA) with dextran, an excipient (as a thickener) (Solution “B”). The device is provided sterile, ready for use, in pre-filled syringes packaged in single peel Tyvek-polyethylene pouches to provide up to 1.5 mL of the adhesive composition.

Each solution (approximately 1.0 mL) is filled into a separate chamber of a dual-chamber syringe that is sealed with a dual-opening cap at the tip and with “piston” stopper-seals at the end of each chamber. Each filled and sealed dual-chamber syringe is packaged with a separately provided Vent Adjustment Device (VAD) mixing tip, dual-rod plunger in a sealed single Tyvek-polyethylene peel pouch that is terminally sterilized by radiation.

The “piston” stopper-seals are designed to accommodate the ends of the dual-rod plunger that is inserted into the end of each chamber and advanced to engage the “piston” stopper seals.

The VAD mixing tip is designed to be attached to the tip of the dual-chamber syringe (in place of the cap) and both: (1) mechanically purges the dead-space air from the hub and mixing element segment of the mixing tip through its vent feature; (2) helps to ensure a 1:1 mixture of the two solutions; and (3) mixes the two solutions as they are dispensed.

The adhesive composition is prepared for use by removing the cap at the tip of the dual-chamber syringe (twist-off); affixing the air-bleed VAD mixing tip (twist-on); inserting dual-rod end of the plunger into the ends of the dual-chamber syringe and advancing to contact the back of the “piston” stopper-seals. Optionally a needle may be attached to the end of the VAD mixing tip. For example, the needle can be a commercially available standard injection needle, biopsy needle, or introducer etc., that have a standard luer lock hub of <21 gauge (>0.76 mm ID). The needle can be a 21-18 G standard or steerable Seeker Biopsy Needle(s).

Once the dual-chamber syringe is assembled, the solutions are mixed and dispensed by applying smooth and continuous pressure to the end of the plunger until the desired amount of the adhesive composition is delivered to the tissue/surgical location.

The adhesive composition polymerizes into a solid within 10-50 seconds after it is mixed/dispensed. During the polymerization, the processed glutaraldehyde in the adhesive composition will also cross-link to the tissue site ensuring that the marker remains at the placement site. The polymerized marker is slowly resorbable by hydrolysis.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. An adhesive composition, comprising:
a protein component having a first viscosity; and an aldehyde component having a second viscosity, wherein the aldehyde component further comprises a thickening agent.

2. The composition of claim 1, wherein the protein component is derived from a mammalian source.

3. The composition of claim 2, wherein the mammalian source is selected from the group consisting of human, bovine, bison, ovine, and porcine.

4. The composition of claim 1, wherein the protein component is derived from a recombinant source.

5. The composition of claim 1, wherein the protein component is a serum albumin.

6. The composition of claim 1, wherein the protein component comprises more than 15% protein monomer.

7. The composition of claim 1, wherein the aldehyde component is a dialdehyde or a polyaldehyde.

8. The composition of claim 7, wherein the dialdehyde is a glutaraldehyde.

9. The composition of claim 1, wherein the protein component is a serum albumin and the aldehyde component is a glutaraldehyde.

10. The composition of claim 1, wherein the thickening agent increases the second viscosity of the aldehyde component by at least about 5%.

11. The composition of claim 1, wherein the thickening agent is selected from the group consisting of dextran, carboxymethyl cellulose, polyethylene glycol, liposomes, proliposomes, glycerol, starch, carbohydrates, povidone, polyethylene oxide, and polyvinyl alcohol.

12. The composition of claim 1, wherein the thickening agent is dextran.

13. The composition of claim 1, wherein the thickening agent comprises at least about 0.5% of the composition.

14. The composition of claim 1, wherein the thickening agent alters a gel time of the composition.

15. The composition of claim 1, wherein the composition further comprises a radiopaque material.

16. The composition of claim 15, wherein the radiopaque material is selected from the group consisting of bismuth oxide (Bi₂O₃), zinc oxide (ZnO), barium sulfate (BaSO₄) lanthanum oxide (La₂O₃), cerium oxide (CeO₂), terbium oxide, ytterbium oxide, neodymium oxide, zirconia (ZrO₂), strontia (SrO), tin oxide (SnO₂), radiopaque glass and silicate glass.

17. An adhesive composition, comprising: a protein component having a first viscosity; and an aldehyde component having a second viscosity, wherein the composition comprises between about 1-26% protein concentration.

18. The composition of claim 17, wherein the protein component comprises more than 15% protein monomer.

19. The composition of claim 17, wherein the protein component comprises between about 1-75% protein concentration.

20. The composition of claim 17, wherein the composition is comprises of between 1-75% protein concentration.

21. The composition of claim 17, wherein the protein component and the aldehyde component are present in a ratio of from 6:1 to 1:6.

22. The composition of claim 17, wherein the protein component and the aldehyde component are present in the ratio of 1:1.

23. An adhesive composition comprising a mixture of: a serum albumin component having a first viscosity; and an aldehyde component having a second viscosity, wherein the composition comprises between about 1-26% of the serum albumin concentration.

24. The composition of claim 23, wherein the protein component comprises more than 15% protein monomer.

25. The composition of claim 23, wherein the serum albumin component is derived from porcine.

26. The composition of claim 23, wherein the aldehyde component is a glutaraldehyde.

27. The composition of claim 23, wherein the aldehyde composition further comprises a thickening agent.

28. The composition of claim 27, wherein the thickening agent is dextran, polyethylene glycol or carboxymethyl cellulose.

29. The composition of claim 23, wherein the second viscosity of the aldehyde component approximates the first viscosity of the serum albumin component.

30. The composition of claim 23, wherein the serum albumin component comprises between about 1-75% serum albumin concentration.

31. The composition of claim 23, wherein the serum albumin component and the aldehyde component are present in a ratio of from 6:1 to 1:6.

32. A method for causing adhesion of tissue, comprising:
(i) mixing a first composition comprising a protein component having a first viscosity with a second composition comprising an aldehyde component having a second viscosity to obtain an adhesive composition and adjusting the second viscosity of the second composition with a thickening agent;
(ii) introducing the adhesive composition to a tissue; and
(iii) allowing the adhesive composition to adhere to the tissue.

33. The composition of claim 32, wherein the protein component comprises more than 15% protein monomer.

34. The method of claim 32, wherein the protein component is derived from porcine.

35. The method of claim 32, wherein the protein component is a serum albumin.

36. The method of claim 32, wherein the aldehyde component is a dialdehyde or a polyaldehyde.

37. The method of claim 32, wherein the aldehyde component is a glutaraldehyde.

38. The method of claim 32, wherein the protein component is a serum albumin and the aldehyde component is a glutaraldehyde.

39. The method of claim 32, wherein the first viscosity approximates the second viscosity.

40. The method of claim 32, wherein the first viscosity matches the second viscosity.

41. The method of claim 32, wherein the thickening agent is selected from the group consisting of dextran, carboxymethyl cellulose, polyethylene glycol, liposomes, proliposomes, glycerol, starch, carbohydrates, povidone, polyethylene oxide, and polyvinyl alcohol.

42. The method of claim 32, wherein the thickening agent is dextran.

43. The method of claim 32, wherein the thickening agent comprises at least about 0.5% of the adhesive composition.

44. The method of claim 32, wherein the thickening agent alters a gel time of the adhesive composition.

45. The method of claim 32, wherein the protein component is about 5-75% of the composition.

46. The method of claim 32, wherein the protein component and the aldehyde component are present in a ratio of 6:1 to 1:6 in the final composition.

47. The method of claim 32, wherein the composition further comprises a radiopaque material.

48. The composition of claim 47, wherein the radiopaque material is selected from a group consisting of bismuth oxide (Bi₂O₃), zinc oxide (ZnO), barium sulfate (BaSO₄) lanthanum oxide (La₂O₃), cerium oxide (CeO₂), terbium oxide, ytterbium oxide, neodymium oxide, zirconia (ZrO₂), strontia (SrO), tin oxide (SnO₂), radiopaque glass and silicate glass.

49. A method for marking a location within the human body, comprising:
(i) providing a first component comprising a protein having a first viscosity and a second component comprising an aldehyde having a second viscosity;
(ii) mixing the first component with the second component resulting in a target composition;
(iii) introducing the target composition to a location; and
(iv) marking a site of the location with the target composition.

50. The method of claim 49, wherein the target composition is a palpable material.

51. The method of claim 49, wherein the protein is a serum albumin derived from porcine.

52. The method of claim 49, wherein the aldehyde is a glutaraldehyde.

53. The method of claim 49, wherein the first viscosity of the first component or the second viscosity of the second component is altered prior to the mixing step.

54. A process of making an adhesive composition, comprising:
(i) providing a first protein component with a first viscosity;
(ii) providing a second aldehyde component with a second viscosity;
(iii) adjusting the first viscosity, the second viscosity or both; and
(iv) mixing the first component with the second component to obtain an adhesive composition.

55. The composition of claim 54, wherein the protein component comprises more than 15% protein monomer.

56. The process of claim 54, wherein the protein component is a serum albumin derived from porcine.

57. The process of claim 54, wherein the aldehyde component is a glutaraldehyde.

58. The process of claim 54, wherein the first viscosity of the first component approximates the second viscosity of the second component.
